(19) United States
(12) Patent Application Publication
(10) Pub. No.: US 2016/0051419 A1
(43) Pub. Date: Feb. 25, 2016

(54) ABSORBENT ARTICLE WITH GRAPHICS

(71) Applicant: The Procter & Gamble Company, Cincinnati, OH (US)

(72) Inventors: Matthew Howard Wasson, Cincinnati, OH (US); Miguel Alvaro Robles, Wyoming, OH (US); Nicholas Edward Gressle, Cincinnati, OH (US)

(21) Appl. No.: 14/832,138

(22) Filed: Aug. 21, 2015

Related U.S. Application Data
(60) Provisional application No. 62/040,271, filed on Aug. 21, 2014.

(51) Int. Cl. A61F 13/472; A61F 13/84

(52) U.S. Cl. CPC A61F 13/472; A61F 13/84; A61F 2013/15243

(57) ABSTRACT

The present application relates to absorbent articles, including catamenial devices, such as tampons and sanitary napkins, for the absorption of menses. More particularly, the present invention relates to catamenial devices comprising one or more graphical elements wherein the graphical elements highlight the functional area of the absorbent article.

### FIELD OF INVENTION

The present application relates to absorbent articles, including catamenial devices, such as sanitary napkins, for the absorption of menses. More particularly, the present invention relates to catamenial devices comprising one or more graphical images wherein the graphical images outline the functional area of the absorbent article.

### BACKGROUND OF THE INVENTION

Disposable absorbent articles, such as sanitary napkins, diapers, adult incontinence, and catamenial pads are manufactured at a high rate of speed. However today's modern high speed production lines have an inherent variation in the cross direction (CD) tracking of webs and the combining of webs. If noticed by the consumer, this CD variation can impact the quality perception of the product. For example, a consumer may believe that a product will not work properly if they notice that the absorbent core is not centrally located between the wing components-of the article.

In addition, consumers like color. Color is often added in the form of graphics or graphical elements to a layer in the product. Printing graphical elements on the top layer of the article can communicate a barrier signal or highlight the functional area of the product. However, printed product graphical elements, like barrier signals to highlight the functional area of the product, that are consumer preferred can actually accentuate the perception of CD variation found in today's products, making the consumers believe that the article is defective, when in fact the product is still within quality standards to function properly. To deal with this problem, current products either sacrifice an effective barrier signal graphic or are forced to incur large expenses to slow down the lines or purchase specialized equipment to reduce the CD variation. These costs can be prohibitive.

For example, current products have printed far away from the perimeter of the core so that the core placement perception will be more acceptable for the large CD variation. Graphical elements have also been placed on the entire backsheet before the placement of the core to not have to be concerned with the CD variation. Lastly, graphical elements have been placed in an asymmetric fashion in areas outside of the core perimeter to avoid dealing with the CD variation.

As such, what is needed is a specific printed graphic orientation on the topsheet of an article that enables a consumer acceptable signal and is capable of being created in a high speed manufacturing process.

### SUMMARY OF THE INVENTION

An absorbent article having a liquid pervious topsheet, a backsheet joined to the topsheet, and an absorbent core having a perimeter disposed between the topsheet and the backsheet. The topsheet has an inner surface oriented toward the interior of the absorbent article or garment facing side and an outer surface oriented toward the skin of the wearer when the absorbent article is being worn or body facing side. The backsheet has an inner surface oriented toward the interior of the absorbent article and an outer surface oriented toward the garment of the wearer when the absorbent article is being worn. The absorbent core has an inner surface oriented toward the skin of the wearer when the absorbent article is being worn and an outer surface oriented toward the garment of the wearer when the absorbent article is being worn. The topsheet has four or more graphical elements. The graphical elements overlap the perimeter of the absorbent core such that the core perimeter exhibits an ink to clean ratio of between 5% to 60%.

An absorbent article having a liquid pervious topsheet, a backsheet joined to the topsheet, and an absorbent core having a perimeter disposed between the topsheet and the backsheet. The topsheet has an inner surface oriented toward the interior of the absorbent article or garment facing side and an outer surface oriented toward the skin of the wearer when the absorbent article is being worn or body facing side. The backsheet has an inner surface oriented toward the interior of the absorbent article and an outer surface oriented toward the garment of the wearer when the absorbent article is being worn. The absorbent core has an inner surface oriented toward the skin of the wearer when the absorbent article is being worn and an outer surface oriented toward the garment of the wearer when the absorbent article is being worn. A functional layer signal generally centrally located on a layer beneath the topsheet. The topsheet has four or more graphical elements. The graphical elements overlap the perimeter of the absorbent core such that the core perimeter exhibits an ink to clean ratio of between 5% to 60%.

### BRIEF DESCRIPTION OF THE DRAWINGS

While the specification concludes with claims particularly pointing out and distinctly claiming the subject matter of the present invention, it is believed that the invention may be more readily understood from the following description taken in connection with the accompanying drawings, in which:

FIG. 1 is a top view of a representative catamenial device.

FIG. 2 is a product graphic arrangement as described.

FIGS. 3A-C show a product graphic arrangement as described.

FIGS. 4-7 represent product graphic arrangements within the scope of the invention.

### DETAILED DESCRIPTION OF THE INVENTION

The present invention is directed to printed product graphics and to absorbent articles, particularly disposable absorbent articles, having one or more graphics visible from the body facing side of the topsheet. The product graphic arrangement allows for high speed manufacturing while highlighting the functional zone of the article. Disposable absorbent articles can be feminine hygiene articles, including incontinence devices and catamenial products, such as, sanitary napkins, pantiliners, interlabial products, and the like. For convenience, the invention is disclosed below with respect to the embodiment of a catamenial device, such as a sanitary napkin or pantiliner.

As used herein, “visible ink to clean area percentage” relates to the percentage of the absorbent core perimeter that is overlapped by a graphic. The visible ink to clean area percentage can be calculated by measuring the absorbent core perimeter, measuring the portions of any graphical elements that overlap the absorbent core perimeter along the absorbent core perimeter, summing up the total length of the absorbent core perimeter that is overlapped by graphical elements, and dividing the sum of the absorbent core perimeter that is overlapped by graphical elements by the total absorbent core perimeter.

As used herein, a “functional layer signal” relates to any identifier that draws attention to the functional absorbent portions of the absorbent article. The functional layer signal can be a structural identifier such as, for example, apertures in the form of slots or dots in or around the functional layer, graphical elements on the viewing portion of the functional layer or around the functional layer, or a combination of structural elements and graphical elements that direct a user to the functional layer of the absorbent article.

As user herein, a “printed graphic orientation” relates to a set of graphical elements within a given area. However, as mentioned, the graphical elements need not be printed on the same surface within this area.

As used herein, a “product graphic arrangement” relates to the combination of graphical elements visible from the body side surface of the absorbent article. The product graphic arrangement can have four or more graphical elements. The product graphic arrangement can be a functional layer signal.

Regardless of the particular configuration of the layers used in forming the absorbent article, a plurality of graphical elements are printed in a product graphic arrangement so that they are visible from the body-facing surface. The particular graphical elements are selected to provide a unique visual appearance that highlights the functional zone to the consumer while allowing for high speed manufacturing of the absorbent article. High speed manufacturing is done by placing one layer of material on top of another layer until the article is complete. Each layer of material must be placed within a calculated margin of error. Otherwise, the manufacturing system can deem that the product is unacceptable. Although a product can be acceptable due to the manufacturing guidelines, it is possible to have a sanitary napkin wherein the absorbent core has shifted in the CD direction along the transverse axis. The inventors have found that by placing the graphical elements in a unique printed graphic orientation, the sanitary napkin can have consumer acceptable graphical elements that highlight the functional zone while allowing for high speed manufacturing.

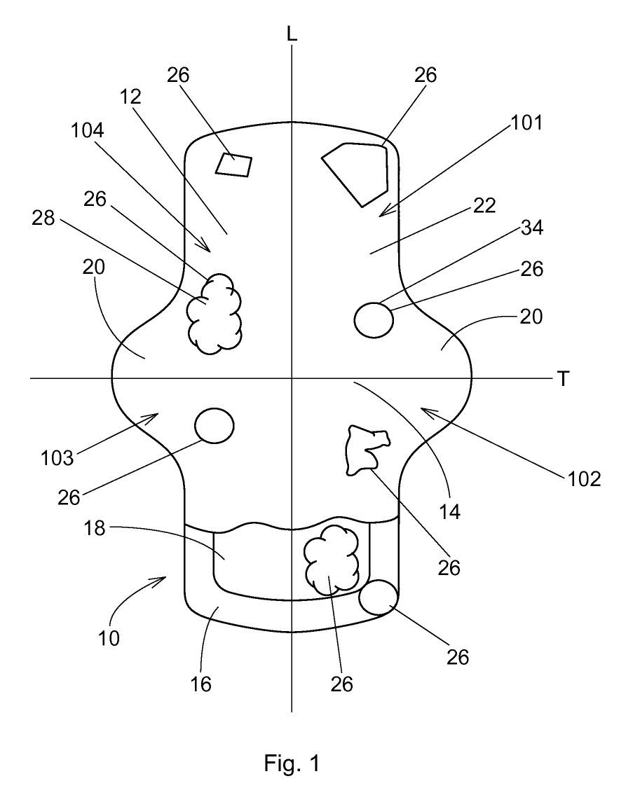

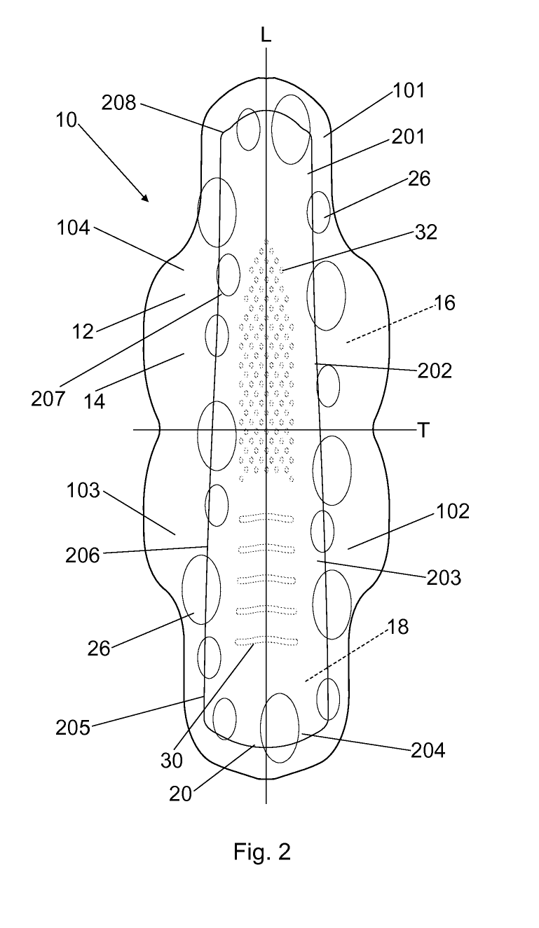

FIG. 1 shows a sanitary napkin 10, e.g., having a body-contacting surface 12 comprising an outer surface of a topsheet 14, a liquid impervious backsheet 16 joined to the topsheet 14, and an absorbent core 18. The sanitary napkin 10 has a longitudinal axis L, a transverse axis T. The longitudinal axis and the transverse axis meet at a central point of the sanitary napkin. The longitudinal axis and transverse axis break the sanitary napkin into four quadrants 101, 102, 103, 104. The absorbent core has a perimeter. The absorbent core perimeter of the absorbent core 20 can be broken up into octants. An octant is calculated by measuring the length of the absorbent core perimeter and dividing the total length by eight. As shown in FIG. 2, each octant contains an equal length of the absorbent core perimeter 201, 202, 203, 204, 205, 206, 207, and 208. Each quadrant contains a portion of the absorbent core inclusive of the absorbent core perimeter.

Graphical elements 26 can be printed on the body facing surface or the garment facing surface of the topsheet so that they are readily visible. Additional graphical elements 26 can be printed on any layer of the absorbent article 10. Additional graphical elements 26 can be printed on the body facing side of the backsheet 16, on the body facing side of a secondary topsheet (not shown), on a body facing side of an absorbent core 18, or on a portion of a body facing side of the absorbent core 18.

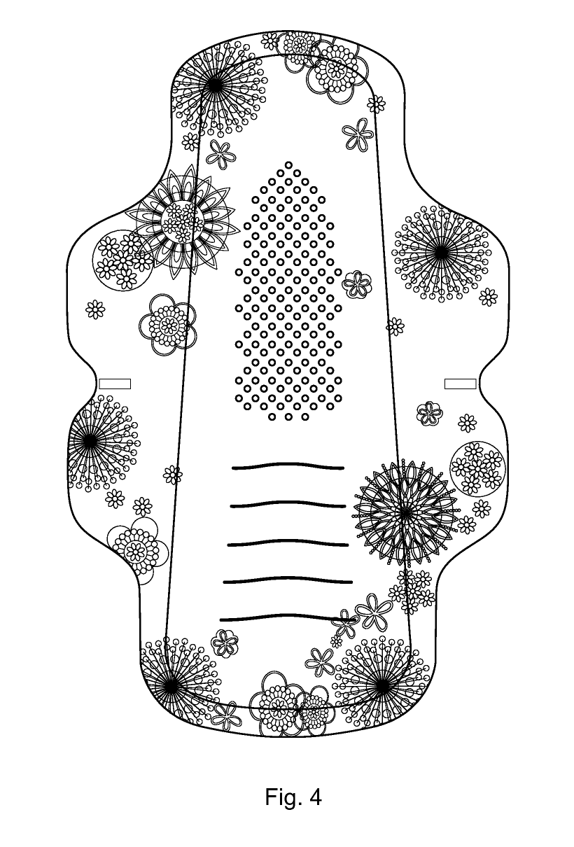

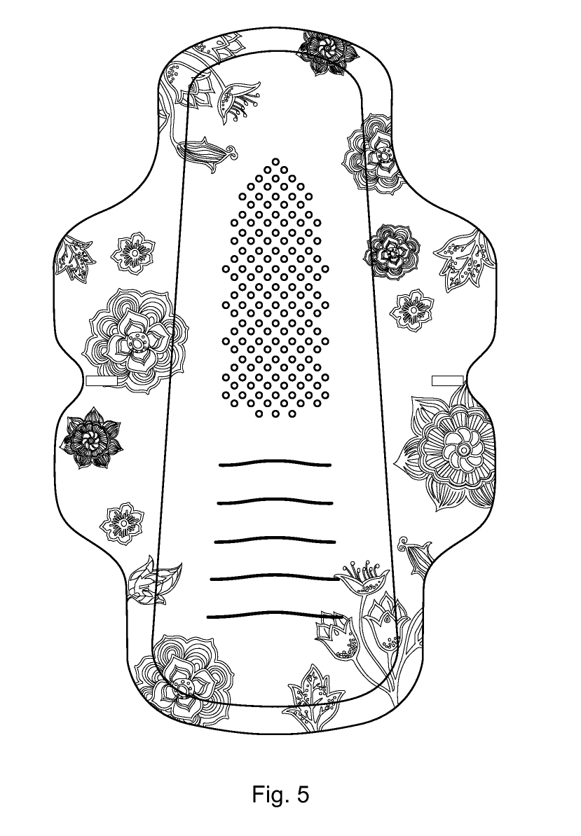

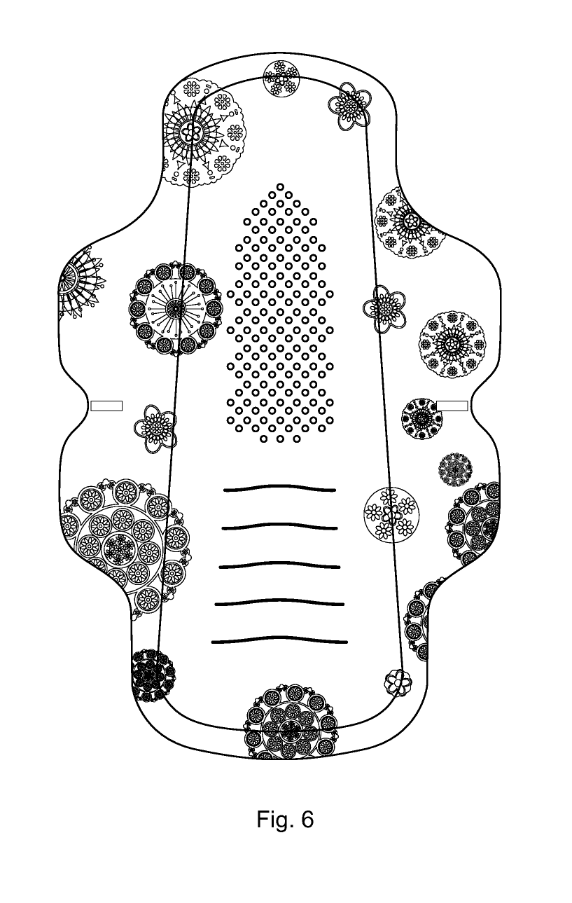

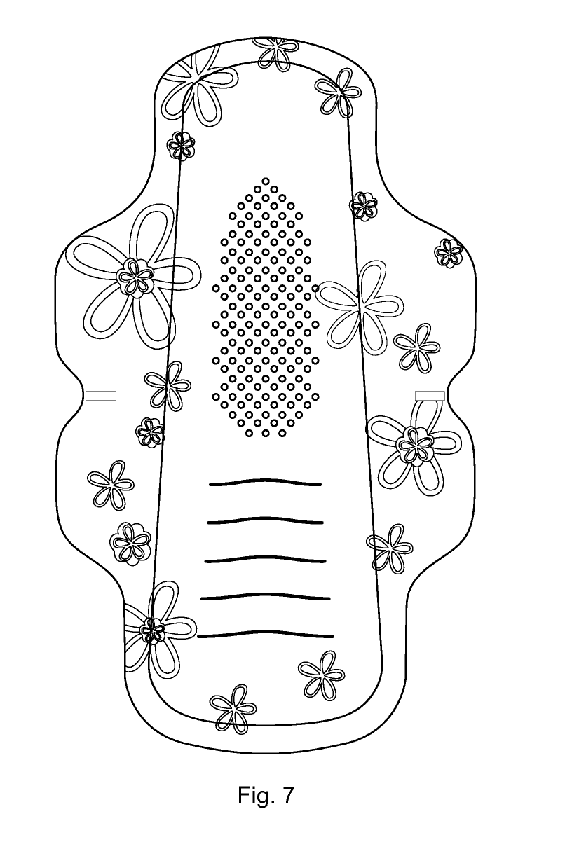

The graphical elements can have different sizes, colors, shapes, and designs. The graphical elements can be balanced to the extent that several smaller graphical elements on one side can be “balanced” by a large graphical element on the other side, or smaller objects can be placed further away from the center than larger objects. Alternatively, a darker object can be similarly balanced by several lighter objects. It also should be noted that although the graphical elements 28 and 34 are shown herein as possessing relatively simple shapes, this is merely for exemplary purposes. Virtually any shape or design can be employed, such as dots, ovals, triangles, squares, rectangles, flowers, butterflies, stars, hearts, spirals, double spirals, clothold curves, comu spirals, polynomial double spirals, Euler's double spirals, parametric double spirals, etc. In fact, it is generally preferable that the graphical elements are more complicated in nature and that they possess some degree of curvature and color to provide a more complex visual perception and aid in the overall masking effect of the article. For example, FIGS. 4-7 generically illustrate more complex graphical elements that can be employed in the present invention.

In certain cases, the graphical elements can also be arranged to impart an optical illusion when viewed from a particular focal point of the user. For instance, the arrangement of the graphical elements relative to one another can form an optical illusion of motion perceivable as at least one of the objects appearing to move away from an edge of the body. Exemplary objects for providing such an illusionary pattern can include geometric, spiral, anomalous motion, rotational, color change, peripheral drift, positive after image blurs, scintillation grid, stereopsis and chromosteropsis, contraction and expansion, contrast polarities, convection, chromatic assimilation, etc., such as described in U.S. Patent Publication Nos. 2009/0157021 to Sullivan, et al. and 2005/0268371 to Meekins, which are incorporated herein in their entirety by reference thereto for all purposes. In many embodiments, graphical elements of different shades can be employed to create a perception of depth within the absorbent article by a user looking upon the viewing surface of the topsheet. Such illusionary patterns are described in U.S. Patent Publication No. 2003/0114811 to Christen, et al., which is incorporated herein in its entirety by reference thereto for all purposes.

The printed graphic orientation highlights the absorbent core or functional area of the article while allowing for flexibility during high speed manufacturing. The inventors have found that by balancing the location of the graphical elements, by placing a size limitation on the graphical elements, and by placing graphical elements along the absorbent core perimeter to have a visible ink to clean area percentage of 5% to 60% along the core perimeter, one can achieve a product graphic orientation that allows for high speed manufacturing while being consumer acceptable.

In many embodiments, the functional area can have an additional functional indicator within the perimeter of the absorbent core. The additional functional indicator can be one or more additional graphical elements, apertures, perforations, slots, or dots in the core. The additional functional indicator can be located under the topsheet, in or on the absorbent core, or any other layer that is visible from the body facing surface of the topsheet.

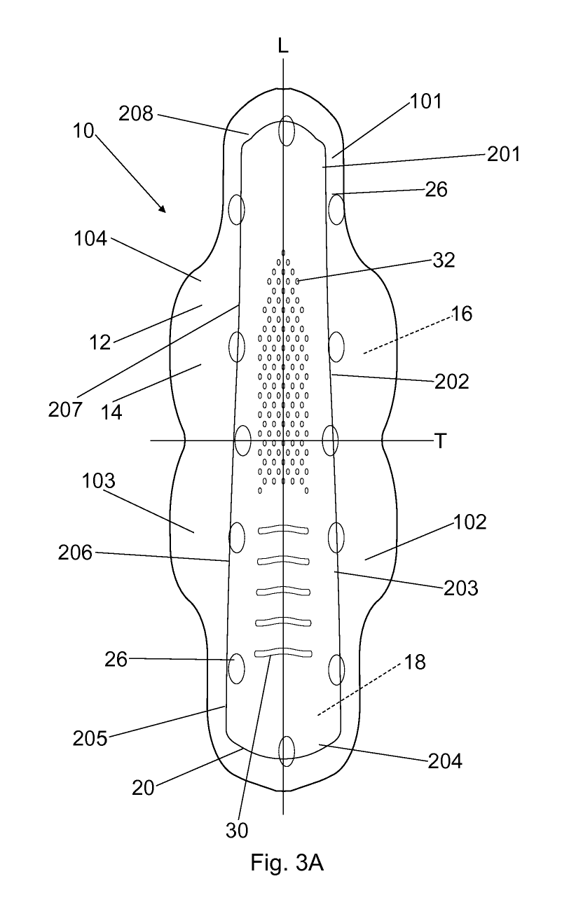

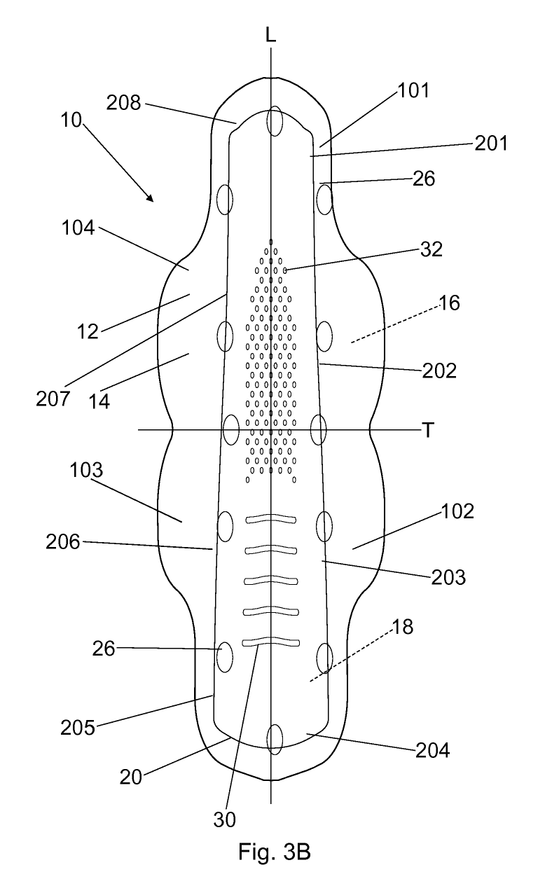

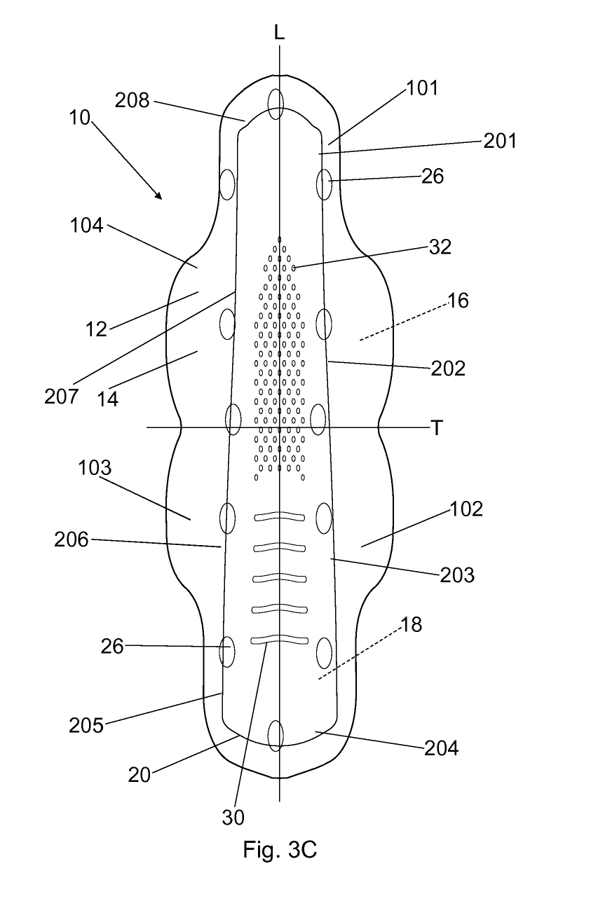

As shown in FIGS. 2 and 3A-C, the inventors have found that graphical elements can be placed to meet consumer acceptability while allowing for high speed manufacturing and highlighting the functional zone of the absorbent article. Without being bound by theory, the inventors have found that the objectives are achieved by following a recommended criteria for the overall product graphic arrangement.

FIGS. 2 and 3A-C show product graphic arrangements on an absorbent article 10. The absorbent article 10 has a topsheet 14, a backsheet 16, a body facing surface 12, and an absorbent core 18. The product graphic arrangements comprise of four or more graphical elements 26. The absorbent article 10 is broken up into quadrants 101, 102, 103, 104 by the longitudinal axis and transverse axis. The absorbent core perimeter 20 is broken up into octants 201, 202, 203, 204, 205, 206, 207, and 208. As shown in FIGS. 2 and 3A-C, the functional zone contains additional functional layer signals in the form of slots 30 and dots 32. As shown in FIGS. 2 and 3A-C, adjacent graphical elements 26 do not contact or overlap. As shown in FIGS. 2 and 3A-C, no two adjacent graphical elements within a quadrant share a longitudinal center line parallel to the longitudinal axis. As shown in FIGS. 2 and 3A-C, at least one graphic is located in each quadrant. As shown in FIGS. 2 and 3A-C, at least one graphic is located in each of the eight octants.

In many embodiments, the graphical elements along the absorbent core perimeter can not lie along the same line. For example, the graphical elements should not form a straight line. The graphical elements should not contact or make a continuous frame around the absorbent core perimeter.

In many embodiments, the graphical elements are spaced such that there is between 1 mm and 30 mm between each graphic, such as, for example, between 2 mm and 15 mm, between 5 mm and 10 mm, such as, for example, 3 mm, 4 mm, 6 mm, 7 mm, 8 mm, or 9 mm. The distance between graphical elements can vary. For example the distance between a first graphical element and a second graphical element can be 3 mm while the distance between the first graphical element and a third graphical element can be 2.2 mm.

As shown in FIG. 2, the absorbent core 18 can be broken up into quadrants 101, 102, 103, and 104.

The graphical elements 26 can be balanced by placing graphical elements 26 around the perimeter 20 of the absorbent core 18 so that the percentage of ink area along the absorbent core perimeter is between 5% and 60%, between 10% and 50%, such as, for example, 15%, 20%, 25%, 30%, 35%, 40%, 45%, or 50%. As shown in FIG. 2, the visible ink to clean area percentage for the perimeter is about 60%. The graphical elements 26 can be placed in each quadrant provided that they are within the required size restriction of less than 12 cm wide along the CD direction. In many embodiments, the graphic elements vary in size. The graphical elements along the core perimeter should be between 0.1 cm and 12 cm in width. This range allows for the acceptable range for core placement in the CD direction.

As shown in FIGS. 3A-C, the absorbent article 10 have multiple graphical elements 26. The graphical elements 26 can be balanced by placing graphical elements 26 around the perimeter 20 of the absorbent core 18 so that the visible ink to clean area percentage for the perimeter is between 5% and 60%, between 10% and 50%, such as, for example, 15%, 20%, 25%, 30%, 35%, 40%, 45%, or 50%. As shown in FIGS. 3A-C, the product graphic arrangement can be shifted by between 0 and 6 cm from the longitudinal centerline while still highlighting the absorbent core 18 of the absorbent article 10. As shown in FIG. 3A, the product graphic arrangement can be centered to the longitudinal axis and transverse axis. As shown in FIG. 3B, the product graphic arrangement has shifted in both the CD and MD direction. Similarly, as shown in FIG. 3C, the product graphic arrangement has been shifted in both the CD and MD direction. The graphical elements 26 can be placed in each octant provided that they are within the required size restriction of between 0.1 cm and 12 cm in width. In many embodiments, the graphical elements vary in size. It is not required that each octant includes a graphic 26 to highlight the absorbent core 18. A graphic 26 can overlap octants.

The sanitary napkin can also be provided with additional features commonly found in napkins, including “wings” or “flaps” as is known in the art, and/or a secondary topsheet, and/or a fluid acquisition layer, and/or other layers designed to promote fluid transport to absorbent core 18. Likewise, topsheet 14 of the sanitary napkin can have various optional characteristics, as is known in the art. For example, topsheet 14 can have one or more of channels embossed therein to direct fluid flow, apertures therethrough to aid in fluid acquisition, and printed signals visible on or through, the visible signals being printed on the topsheet or underlying layers for functional and esthetic properties.

The topsheet can be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be included of natural fibers (e.g., wood or cotton fibers), synthetic fibers (e.g., polymeric fibers such as polyester, polypropylene, or polyethylene fibers) or from a combination of natural and synthetic fibers. In many embodiments, the topsheet can be made of a hydrophobic material to isolate the wearer's skin from liquids which have passed through the topsheet. If the topsheet is made of a hydrophobic material, at least the upper surface of the topsheet is treated to be hydrophilic so that liquids will transfer through the topsheet more rapidly. This diminishes the likelihood that body exudates will flow off the topsheet rather than being drawn through the topsheet and being absorbed by the absorbent core. In many embodiments, the topsheet can be rendered hydrophilic by treating it with a surfactant. Suitable methods for treating the topsheet with a surfactant include spraying the topsheet material with the surfactant and immersing the material into the surfactant.

The topsheet can include an apertured formed film. Apertured formed films can be used for the topsheet because they are pervious to body exudates and yet non-absorbent and have a reduced tendency to allow liquids to pass back through and rewet the wearer's skin. Thus, the surface of the formed film which is in contact with the body remains dry, thereby reducing body soiling and creating a more comfortable feel for the wearer. Suitable formed films are described in U.S. Pat. No. 3,929,135, entitled “Absorptive Structures Having Tapered Capillaries”, issued to Thompson on Dec. 30, 1975; U.S. Pat. No. 4,324,246 entitled “Disposable Absorbent Article Having A Stain Resistant Topsheet”, issued to Mullane, et al. on Apr. 13, 1982; U.S. Pat. No. 4,342,314 entitled “Resilient Plastic Web Exhibiting Fiber-Like Properties”, issued to Radel, et al. on Aug. 3, 1982; U.S. Pat. No. 4,463,045 entitled “Macroscopically Expanded Three-Dimensional Plastic Web Exhibiting Non-Glossy Visible Surface and Cloth-Like Tactile Impression”, issued to Ahr, et al. on Jul. 31, 1984; and U.S. Pat. No. 5,006,394 “Multilayer Polymeric Film” issued to Baird on Apr. 9, 1991.

The absorbent core can be any absorbent member which is generally compressible, conformable, non-irritating to the wearer's skin, and capable of absorbing and retaining body fluids. The absorbent core can be manufactured in a wide variety of sizes and shapes (e.g., rectangular, hourglass, “T”-shaped, asymmetric, etc.) and from a wide variety of liquid-absorbent materials commonly used in disposable pull-on garments and other absorbent articles such as comminuted wood pulp which is generally referred to as airfelt. Examples of other suitable absorbent materials include creped cellulose wadding; meltblown polymers including coform; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials.

The configuration and construction of the absorbent core can vary (e.g., the absorbent core can have varying caliper zones, a hydrophilic gradient, a superabsorbent gradient, or lower average density and lower average basis weight acquisition zones; or can include one or more layers or structures). Further, the size and absorbent capacity of the absorbent core can also be varied to accommodate wearers ranging from infants through adults. However, the total absorbent capacity of the absorbent core should be compatible with the design loading and the intended use of the absorbent article.

The absorbent core can include other optional components. One such optional component is the core wrap, i.e., a material, typically but not always a nonwoven material, which either partially or totally surrounds the core. Suitable core wrap materials include, but are not limited to, cellulose, hydrophilically modified nonwoven materials, perforated films and combinations thereof.

The backsheet can comprise a liquid impervious film. The backsheet can be impervious to liquids (e.g., body fluids) and can be typically manufactured from a thin plastic film. However, typically the backsheet can permit vapours to escape from the disposable article. In many embodiments, a microporous polyethylene film can be used for the backsheet. A suitable microporous polyethylene film is manufactured by Mitsui Toatsu Chemicals, Inc., Nagoya, Japan and marketed in the trade as PG-P.

One suitable material for the backsheet can be a liquid impervious thermoplastic film having a thickness of from about 0.012 mm (0.50 mil) to about 0.051 mm (2.0 mils), for example including polyethylene or polypropylene. Typically, the backsheet can have a basis weight of from about 5 g/m² to about 35 g/m². However, it should be noted that other flexible liquid impervious materials can be used as the backsheet. Herein, “flexible” refers to materials which are compliant and which will readily conform to the general shape and contours of the wearer's body.

The backsheet can be typically positioned adjacent the outer-facing surface of the absorbent core and can be joined thereto by any suitable attachment device known in the art. For example, the backsheet can be secured to the absorbent core by a uniform continuous layer of adhesive, a patterned layer of adhesive, or an array of separate lines, spirals, or spots of adhesive. Illustrative, but nonlimiting adhesives, include adhesives manufactured by H. B. Fuller Company of St. Paul, Minn., U.S.A., and marketed as HL-1358J. An example of a suitable attachment device including an open pattern network of filaments of adhesive is disclosed in U.S. Pat. No. 4,573,986 entitled “Disposable Waste-Containment Garment”, which issued to Minetola et al. on Mar. 4, 1986. Another suitable attachment device including several lines of adhesive filaments swirled into a spiral pattern is illustrated by the apparatus and methods shown in U.S. Pat. No. 3,911,173 issued to Sprague, Jr. on Oct. 7, 1975; U.S. Pat. No. 4,785,996 issued to Ziecker, et al. on Nov. 22, 1978; and U.S. Pat. No. 4,842,666 issued to Werenicz on Jun. 27, 1989. Alternatively, the attachment device can include heat bonds, pressure bonds, ultrasonic bonds, dynamic mechanical bonds, or any other suitable attachment device or combinations of these attachment devices.

The backsheet can be additionally secured to the topsheet by any of the above-cited attachment devices.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Values disclosed herein as ends of ranges are not to be understood as being strictly limited to the exact numerical values recited. Instead, unless otherwise specified, each numerical range is intended to mean both the recited values and any integers within the range. For example, a range disclosed as “1 to 10” is intended to mean “1, 2, 3, 4, 5, 6, 7, 8, 9, and 10.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. An absorbent article comprising:
a liquid pervious topsheet, the topsheet having an inner surface oriented toward the interior of the absorbent article and an outer surface oriented toward the skin of the wearer when the absorbent article is being worn;
a backsheet joined to the topsheet, the backsheet having an inner surface oriented toward the interior of the absorbent article and an outer surface oriented toward the garment of the wearer when the absorbent article is being worn;
an absorbent core having a perimeter disposed between the topsheet and the backsheet, the absorbent core having an inner surface oriented toward the skin of the wearer when the absorbent article is being worn and an outer surface oriented toward the garment of the wearer when the absorbent article is being worn;
wherein the topsheet comprises four or more graphical elements; and
wherein the graphical elements overlap the perimeter of the absorbent core such that the core perimeter exhibits an ink to clean ratio of between 5% to 60%.

2. The absorbent article of claim 1, wherein adjacent graphical elements do not touch.

3. The absorbent article of claim 1, wherein no two adjacent graphical elements within a quadrant share a longitudinal centerline that is parallel to the longitudinal axis.

4. The absorbent article of claim 1, wherein the absorbent article is divided into quadrants by a longitudinal axis and a transverse axis and wherein at least one graphical element is located in each quadrant.

5. The absorbent article of claim 1, wherein the absorbent core has a perimeter broken up into octants and wherein at least one graphical element is located in each octant.

6. The absorbent article of claim 5, wherein the graphical elements overlap the absorbent core perimeter such that the perimeter line bisects one or more graphical elements.

7. The absorbent article of claim 1, wherein the graphical elements have a width of between 0.1 cm and 12 cm.

8. The absorbent article of claim 1, wherein the graphical elements are printed on a garment facing side of the topsheet.

9. The absorbent article of claim 5, wherein a functional layer signal is located within the absorbent core perimeter.

10. The absorbent article of claim 9, wherein the functional layer signal is selected from the group consisting of a graphical element and a structural identifier.

11. An absorbent article comprising:
a liquid pervious topsheet, the topsheet having an inner surface oriented toward the interior of the absorbent article and an outer surface oriented toward the skin of the wearer when the absorbent article is being worn;
a backsheet joined to the topsheet, the backsheet having an inner surface oriented toward the interior of the absorbent article and an outer surface oriented toward the garment of the wearer when the absorbent article is being worn;
an absorbent core having a perimeter disposed between the topsheet and the backsheet, the absorbent core having an inner surface oriented toward the skin of the wearer when the absorbent article is being worn and an outer surface oriented toward the garment of the wearer when the absorbent article is being worn;
a functional layer signal generally centrally located on a layer beneath the topsheet;
wherein the topsheet comprises four or more graphical elements;
wherein the graphical elements overlap the perimeter of the absorbent core such that the core perimeter exhibits an ink to clean ratio of between 5% to 60%.

12. The absorbent article of claim 11, wherein adjacent graphical elements do not touch.

13. The absorbent article of claim 11, wherein no two adjacent graphical elements within a quadrant share a longitudinal centerline that is parallel to the longitudinal axis.

14. The absorbent article of claim 11, wherein the absorbent article is divided into quadrants by a longitudinal axis and a transverse axis and wherein at least one graphical element is located in each quadrant.

15. The absorbent article of claim 11, wherein the absorbent core has a perimeter broken up into octants and wherein at least one graphical element is located in each octant.

16. The absorbent article of claim 15, wherein the graphical elements overlap the absorbent core perimeter such that the perimeter line bisects one or more graphical elements.

17. The absorbent article of claim 11, wherein the graphical elements have a width of between 0.1 cm and 12 cm.

18. The absorbent article of claim 11, wherein the graphical elements are printed on a garment facing side of the topsheet.

19. The absorbent article of claim 15, wherein a functional layer signal is located within the absorbent core perimeter.

20. The absorbent article of claim 19, wherein the functional layer signal is selected from the group consisting of a graphical element and a structural identifier.
